(19) United States
(12) Patent Application Publication
(10) Pub. No.: US 2023/0390544 A1
(43) Pub. Date: Dec. 7, 2023

(54) INTRAVASCULAR BLOOD PUMPS

(71) Applicant: SHIFAMED HOLDINGS, LLC, Campbell, CA (US)

(72) Inventors: Daniel HILDEBRAND, Santa Cruz, CA (US); S. Mostafa GHOREYSHI, Campbell, CA (US); Amr SALAHIEH, Saratoga, CA (US)

(21) Appl. No.: 18/248,420

(22) PCT Filed: Oct. 12, 2021
(86) PCT No.: PCT/US2021/054569

§ 371 Date: Apr. 10, 2023

Related U.S. Application Data
(60) Provisional application No. 63/089,915, filed on Oct. 9, 2020.

(51) Int. Cl. A61M 60/13; A61M 60/216; A61M 60/408; A61M 60/808

(52) U.S. Cl. CPC A61M 60/13; A61M 60/216; A61M 60/408; A61M 60/808

(57) ABSTRACT

Catheter blood pumps that include a collapsible blood conduit, a collapsible scaffold portion, and a taper formed in the collapsible scaffold. The collapsible blood conduit defines a blood lumen. The collapsible scaffold is adapted to provide radial support to the blood conduit. The blood pump also includes one or more impellers. In some embodiments, the one or more impellers can also include a taper. The taper of the blood conduit can match the taper of the one or more impellers.

### CROSS REFERENCE TO RELATED APPLICATIONS

This application claims the benefit of U.S. Provisional Application No. 63/089,915, filed Oct. 9, 2020, incorporated by reference herein.

This application incorporates the following publications by reference herein in their entireties for all purposes: WO 2018/226991; WO2019/094963A1; WO2019/152875A1; WO2020/028537A1; and WO2020/073047A1.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

The disclosure is related to intravascular blood pump and their methods of and manufacture.

A catheter blood pump is provided, comprising a pump portion that includes an impermeable blood conduit and first and second impellers at least partially disposed in the blood conduit, wherein the first and second impellers have first and second average diameters, respectively, that are different.

In some embodiments, the first impeller is a proximal impeller and has an average diameter that is larger than an average diameter of the distal impeller.

In one embodiment, the first impeller is a distal impeller and the second impeller is a proximal impeller, and wherein the distal impeller has a diameter larger than a diameter of the proximal impeller.

In some embodiments, the blood conduit includes a first section and a second section, the first section having a greater average diameter than an average diameter of the second section, wherein the first section at least partially surrounds the one of the first and second impellers with the greater average diameter.

In one embodiment, the blood conduit includes a transition section between the first and second sections, the transition section having a varying diameter between the first and second section average diameters.

In some embodiments, the blood pump further comprises a plurality of expandable proximal struts extending proximally from the blood conduit.

In some embodiments, the blood pump further comprises a plurality of expandable distal struts extending distally from the blood conduit.

In one example, the first impeller has an average diameter that is between 100% and 500% of an average diameter of the second impeller.

In some examples, the first impeller has an average diameter that is from 125% to 400% of an average diameter of the second impeller.

In one embodiment, the blood conduit, first impeller, and second impeller are all configured to be expandable and collapsible.

In some examples, the blood conduit, first impeller, and second impeller are not configured to be expandable and collapsible.

In some embodiments, the first impeller is adapted and configured to be expanded and collapsed, and wherein the second impeller is adapted and configured such that is does not expand and collapse.

In some embodiments, the blood pump further comprises a delivery sheath, the delivery sheath and the second impeller sized so that the delivery sheath is configured to cause the collapse of the second impeller when the delivery sheath is moved distally relative to the second impeller.

In one embodiment, at least one of the first and second impellers, optionally both, includes at least one blade with a diameter that tapers in a tapering region, the tapering region including a location of an impeller greatest diameter, optionally wherein the greatest diameter is at an end of the tapering region.

In some examples, a tip gap between an outer edge of the at least one blade and an inner wall of the blood conduit is constant in the tapering region.

In some embodiments, the first impeller has its largest diameter in an impeller constant diameter region of the impeller, optionally wherein the first impeller is a proximal impeller, and optionally wherein the first impeller is distal impeller.

In another example, at least one of the first and second impellers, optionally both, includes at least one blade with a diameter that tapers in a tapering region, the tapering region including a location or region where an impeller blade is closest to a blood conduit inner wall.

In some embodiments, at least one of the first and second impellers, and optionally both, have a greatest diameter in an impeller constant diameter region.

In some examples, the transition section has a continuously tapering configuration.

In one embodiment, the transition section comprises an outer profile with a configuration adapted to contact tissue and resist distal migration of the pump portion towards a left ventricle.

In some examples, the transition section comprises a shoulder, optionally wherein the shoulder includes one or more bends that include regions of increased curvature of a blood conduit outer profile in the transition section, the transition section transitioning between a larger diameter proximal region and smaller diameter distal region.

In one embodiment, at least of the first and second impellers, optionally both, include a tapering region that includes a midpoint of the impeller, measured along an impeller length.

In some embodiments, the transition section is configured, optionally tapering and increasing in diameter in the distal direction to act as diffuser to blood flow between a distal impeller and a proximal impeller.

A catheter blood pump is provided, comprising a pump portion that includes an expandable and collapsible blood conduit and first and second impellers at least partially disposed in the blood conduit, wherein the first impeller has a greatest diameter that is different than a greatest diameter of the second impeller.

In some embodiments, the blood pump further comprises any of the features described herein.

In some embodiments, the first impeller is a proximal impeller, and the second impeller is a distal impeller.

In one example, at least one of first and second impeller includes a tapering blade region that includes a midpoint (middle) of the impeller, as measured along a length of the impeller.

A method of placing a catheter blood pump across an aortic valve is provided, comprising deploying a pump portion of the catheter blood pump such that a proximal impeller rotational axis is aligned with an aortic valve axis, and a distal impeller rotational axis is off-axis with the proximal impeller rotation axis, the distal impeller rotation axis aligned more with a long axis of a left ventricle than with the axis of the aortic valve, and optionally completely aligned with the long axis of the left ventricle.

A catheter blood pump is provided, comprising a pump portion that includes an expandable and collapsible blood conduit and a first impeller at least partially disposed in the blood conduit, wherein the first impeller has a taper along its length from a first diameter to a second diameter; and wherein the expandable and collapsible blood conduit includes a first impeller section that has a taper that matches the taper of the first impeller.

In some embodiments, the first impeller comprises a proximal impeller.

In other embodiments, the first impeller comprises a distal impeller.

In some examples, the first diameter is larger than the second diameter.

In another embodiment, the first diameter is smaller than the second diameter.

In some embodiments, the expandable and collapsible blood conduit includes a constant diameter section.

In one embodiment, the first impeller is not disposed within the constant diameter section.

In another embodiment, the constant diameter section is distal to the first impeller.

In some embodiments, the constant diameter section is proximal to the first impeller.

### BRIEF DESCRIPTION OF THE DRAWINGS

FIGS. 1A-1C are side views of an exemplary expandable pump portion that includes an expandable impeller housing that includes a scaffold and blood conduit, and a plurality of impellers.

FIGS. 2A-2B illustrate one embodiment of an expandable pump portion that includes a tapered blood conduit and a plurality of impellers.

FIGS. 3A-3B illustrate another embodiment of an expandable pump portion that includes a multi-tapered blood conduit and a plurality of impellers.

FIGS. 4A-4B illustrate an embodiment of an expandable pump portion with a bend in the tapered blood conduit and a plurality of impellers.

FIGS. 5A-5B illustrate one embodiment of an expandable pump portion that includes a tapered blood conduit and a single impeller.

### DETAILED DESCRIPTION

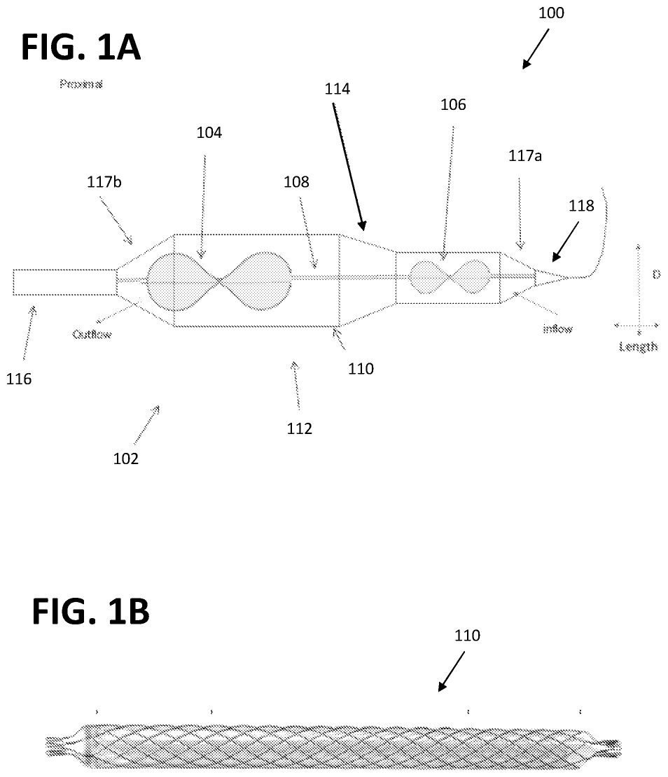

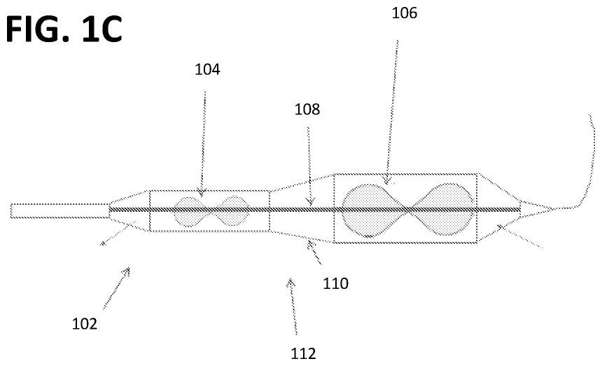

The disclosure is related to catheter blood pumps with a pump that includes a plurality of impellers (for example only, such as shown in FIGS. 1A-1C). One aspect of the disclosure is a pump portion with a first impeller and a second impeller, the first impeller having a greater radially outermost dimension or average dimension than a radially outermost dimension of the second impeller (for example only, as is shown in FIGS. 1A-1C).

Any of the first impellers herein may be a proximal impeller or a distal impeller, and any of the second impellers herein may be a distal impeller or a proximal impeller.

Any of the impellers of this disclosure may have an axial position in the pump portion of the catheter blood pump that might be the same or similar to the axial positions set forth in the Appendix portion of this disclosure. For example, any proximal impeller may extend partially proximally out of an expandable blood conduit, a mere example of which is shown in FIG. 1A, where the proximal impeller extends beyond the blood conduit into a region defined by the proximal struts. For example again, any distal impeller may be disposed completely within an expandable blood conduit, examples of which are shown in FIGS. 1A-1C. Any proximal impeller may alternatively be disposed completely within an expandable blood conduit (e.g., not extending into the region defined by the proximal struts), and alternatively any distal impeller may alternatively extend at least partially distal outside of an expandable blood conduit. Any of the expandable and collapsible blood conduits in this disclosure may include one or more scaffolds or scaffold sections that are coupled to one or more membrane layers.

In some embodiments (an example of which is shown in FIG. 1A), a proximal impeller may have a radially outermost dimension that is greater than a radially outermost dimension of a distal impeller. In some embodiments, a distal impeller has a radially outermost dimension that is greater than a radially outermost dimension of a proximal impeller (an example of which is shown in FIG. 1C).

In any of the embodiments herein, the radially outermost dimension generally refers to a diameter or average diameter of the individual impellers. Unless otherwise indicated, the radially outermost dimension generally refers to the largest dimension of the impeller relative to a rotational axis of the impeller, which may also be a long axis of the pump portion, measured orthogonally relative to the rotational axis. This dimension is generally referred to as a diameter “D” dimension, illustrated in FIG. 1, which may be considered to be orthogonal to an impeller rotational axis and optionally a pump portion long axis. Any of the impellers herein also have an average diameter.

FIG. 1A is a side view illustrating a catheter blood pump 100, including pump portion 102, wherein pump portion 102 includes proximal impeller 104 and distal impeller 106, both of which are in operable communication with drive mechanism 108. Pump portion 102 is in an expanded configuration in FIG. 1A, but is adapted to be collapsed to a delivery configuration so that it can be delivered with a lower profile. In some embodiments, the blood pump can be delivered with a delivery sheath (not shown), the delivery sheath and the impeller(s) being sized so that the delivery sheath is configured to cause the collapse of the one or both of the impellers when the delivery sheath is moved distally relative to the impellers. The impellers can be attached to the drive mechanism 108 (e.g., a drive cable or shaft). Drive mechanism 108 is in operable communication with an external motor, not shown, and extends through or include an elongate shaft. The phrases “pump portion” and “working portion” (or derivatives thereof) may be used herein interchangeably unless indicated to the contrary. For example without limitation, “pump portion” 102 can also be referred to herein as a “working portion.”

Pump portion 102 also includes expandable member or expandable scaffold 110. In this embodiment the scaffold has a proximal end that extends further proximally than a proximal end of the proximal impeller, and a distal end that extends further distally than a distal end of the distal impeller. Expandable members may also be referred to herein as expandable scaffolds or scaffold sections. Expandable scaffold 110 can be disposed radially outside of the impellers along the axial length of the impellers. Expandable scaffold 110 can be constructed in a manner and made from materials similar to many types of expandable structures that are known in the medical arts to be able to collapsed and expanded, examples of which are provided herein. Examples of suitable materials include, but are not limited to, polyurethane, polyurethane elastomers, metallic alloys, etc.

The scaffold 110 can be covered or attached to a cover or membrane to form a blood conduit 112. Any of the blood conduits herein act to, are configured to, and are made of material(s) that create a fluid lumen therein between a first end (e.g., distal end) and a second end (e.g., proximal end). Fluid flows into the inflow region, through the fluid lumen, and then out of an outflow region. Flow into the inflow region may be labeled herein as “Inflow,” and flow out at the outflow region may be labeled “Outflow.” Any of the conduits herein can be impermeable. In some embodiments the conduit is a membrane, or other relatively thin layered member. Any of the conduits herein, unless indicated to the contrary, can be secured to an expandable scaffold such that the conduit, where is it secured, can be radially inside and/or outside of the expandable member. For example, a conduit may extend radially within the expandable member so that inner surface of the conduit is radially within the expandable member where it is secured to the expandable member.

As described above, the blood conduit 112, which is coupled to and supported by the expandable scaffold, has a length L, and extends axially between the impellers. Blood conduit 112 creates and provides a fluid lumen between the two impellers. When in use, fluid moves through the lumen defined by conduit 112. The conduits herein are also flexible, unless otherwise indicated. The conduits herein extend completely around (i.e., 360 degrees) at least a portion of the pump portion. The structure of the expandable scaffold creates at least one inlet aperture to allow for inflow and at least one outflow aperture to allow for outflow. Conduit 112 improves impeller pumping dynamics, compared to pump portions without a conduit. As described herein, expandable members or scaffolds may also be considered to be a part of the blood conduit generally, which together define a blood lumen. In these instances the scaffold and material supported by the scaffold may be referred to herein as an expandable impeller housing or housing.

Expandable scaffolds as described herein may have a variety of constructions, and be made from a variety of materials. For example, expandable scaffold 110 may be formed similar to expandable stents or stent-like devices, or any other example provided herein. For example without limitation, expandable scaffold 110 could have an open-braided construction, such as a 24-end braid, although more or fewer braid wires could be used, an example of which is shown in FIG. 1B. Exemplary materials for the expandable member as well as the struts herein include nitinol, cobalt alloys, and polymers, although other materials could be used.

Expandable scaffold 110 has an expanded configuration, as shown in FIG. 1A, in which the outer dimension (measured orthogonally relative a longitudinal axis of the working portion) of the expandable scaffold 110 is greater in at least a region where it is disposed radially outside impeller 104 than in a transitional region 114 of the expandable scaffold and/or where it is disposed radially outside impeller 106. Drive mechanism 108 is co-axial with the longitudinal axis in this embodiment. In use, the transitional region can be placed across a valve, such as an aortic valve. In some embodiments, expandable scaffold 110 is adapted and constructed to expand to an outermost dimension of 12-24F (4.0-8.0 mm) where the impellers are axially within the expandable member. The smaller transitional region outer dimension and region around impeller 106 can reduce forces acting on the valve, which can reduce or minimize damage to the valve. The larger dimensions of the expandable member in the regions of impeller 104 can help stabilize the working portion axially when in use. Expandable scaffold 110 has an outer configuration that tapers as it transitions from the regions around impeller 104 through the transitional region 114 towards the regions around impeller 106, and again tapers at the distal and proximal ends of expandable scaffold 110 with distal struts 117a and proximal struts 117b.

Expandable scaffold 110 has a proximal end that is coupled to a catheter shaft 116, and a distal end that is coupled to distal tip 118. The impellers and drive mechanism 108 rotate within the expandable scaffold and conduit assembly.

In some embodiments, expandable scaffold 110 can be collapsed by pulling tension from end-to-end on the expandable member. This may include linear motion (such as, for example without limitation, 5-20 mm of travel) to axially extend expandable member 1602 to a collapsed configuration with collapsed outer dimension(s). Expandable member 1602 can also be collapsed by pushing an outer shaft such as a sheath over the expandable member/conduit assembly, causing the expandable member and conduit to collapse towards their collapsed delivery configuration.

Impellers 104 and 106 are also adapted and constructed such that one or more blades will stretch or radially compress to a reduced outermost dimension (measured orthogonally to the longitudinal axis of the working portion). For example without limitation, any of the impellers herein can include one or more blades made from a plastic formulation with spring characteristics, such as any of the impellers described in U.S. Pat. No. 7,393,181, the disclosure of which is incorporated by reference herein for all purposes and can be incorporated into embodiments herein unless this disclosure indicates to the contrary. Alternatively, for example, one or more collapsible impellers can comprise a superelastic wire frame, with polymer or other material that acts as a webbing across the wire frame, such as those described in U.S. Pat. No. 6,533,716, the disclosure of which is incorporated by reference herein for all purposes.

In any of the embodiments herein, a proximal impeller may have a larger diameter or average diameter than a distal impeller diameter or average diameter (examples of which are shown in FIGS. 1A, 2A, 2B, 3A, 3B, 4A and 4B), and the proximal impeller may be configured to generate more pressure than the distal impeller, or do more work than the distal impeller.

In some embodiments, referring to FIG. 1C, a distal impeller may have a larger diameter or average diameter than a proximal impeller, and the distal impeller may be configured to generate more pressure, or do more work, than a proximal impeller.

In any of the embodiments herein, an impeller diameter or average diameter may be from 1 mm-50 mm, such as from 5 mm to 40 mm, or any subrange in either of these ranges.

In any of the embodiments herein, the larger impeller may have a diameter or average diameter that is between 100% (i.e., 1 time) and 500% (i.e., 5 times) of the diameter or average diameter of the relatively smaller impeller, such as from 125% to 400%, or 125% to 300%, or any subrange included within these larger ranges.

In any of the embodiments or claims herein, the impellers and blood conduit may be configured to be expandable and collapsible (examples of which are shown in FIGS. 1A-4B.

In any of the embodiments or claims herein, the one or more impellers and blood conduit may not be configured to be expandable and collapsible. For example, the blood conduit and impellers may have fixed diameter or fixed average diameters.

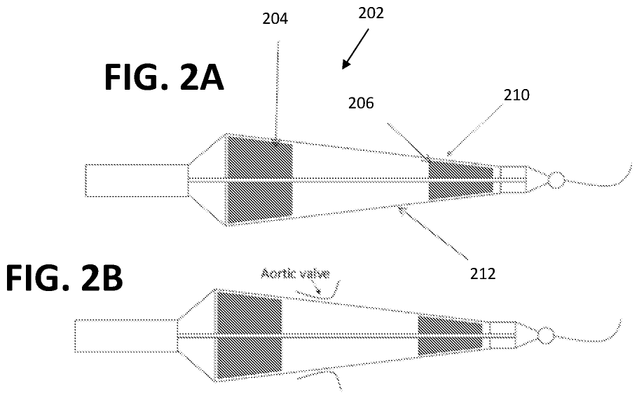

In any of the embodiments herein, the blood conduit may similarly have sections with different diameters or average diameters (mere examples of which are shown in FIGS. 1A-4B). For example, a blood conduit section in which a relatively larger impeller is at least partially disposed may have a larger diameter or average diameter than a blood conduit section in which a relatively smaller impeller is at least partially disposed, such as is shown in FIGS. 1A-4A. For example, in the embodiment in FIG. 1A, the expandable blood conduit illustrates a proximal blood conduit section in which the proximal impeller is at least partially disposed, wherein the proximal blood conduit section has an expanded diameter that is greater than a diameter of a distal blood conduit section in which the distal impeller is disposed. Additionally for example, the embodiments in FIGS. 2A-4B illustrate an expandable blood conduit that includes a proximal blood conduit section in which the proximal impeller is at least partially disposed, wherein the proximal blood conduit section has an expanded average diameter that is greater than an average diameter of a distal blood conduit section in which the distal impeller is disposed.

In various embodiments, a first impeller and corresponding blood conduit section may be non-expandable, and a second impeller and corresponding blood conduit section may be expandable. The different sections may be axially spaced and coupled with a transitional expandable member, such as a transitional expandable member that includes a scaffold coupled to a membrane. By way of example only, in an alternative to FIG. 1C, the distal impeller and distal blood conduit section may be non-expandable, and the transition region, proximal impeller, and proximal blood conduit section may be configured and adapted to be expanded and collapsed.

Any of the impellers herein that include blades that are not tapered (i.e., a greatest diameter section where the diameter is constant) may still be considered to have average diameters in that section. Any of the blood conduits sections herein that are not tapered (i.e., diameter is constant in that section) may still be considered to have average diameters in that section.

Greatest diameter, as that phrase is used herein, refers to a largest diameter dimension of the impeller. It may be constant for some length of the impeller, or it may exist in a tapering blade region, such as at an end of tapering region, for example. The greatest diameter may also be in a region in which a gap between blade and the blood conduit wall is smallest, but this is not necessarily the case, and may depend on the configuration of the blood conduit in that region. For example, an impeller greatest diameter location may be spaced further from a blood conduit than an impeller location that has a diameter less than the greatest diameter.

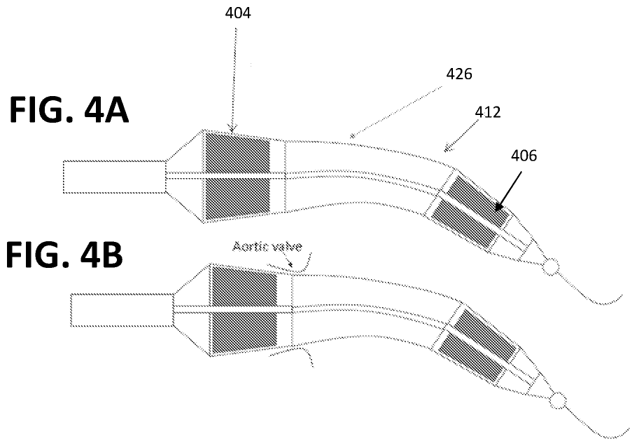

FIGS. 2A, 2B, 3A, 3B, 4A and 4B show examples of impellers that have diameters that taper. In the exemplary FIGS. 2A-4B, the impeller tapers may be in impeller regions where a tip gap between a blade edge and blood conduit wall is smallest and constant (or substantially constant). This description is meant to illustrate a difference between the tapered regions described herein and end regions of impeller that may also include tapers, but in the end regions the tip gap is generally not constant or varies to a greater extent. In this context the impeller tapered regions may be described or considered as being in a central region or more central region of the impeller, which can distinguish between end regions in which the tip gap generally is not constant. The tapered regions herein, however, may abut or be immediately axially adjacent to one or more impeller end regions that may be tapered. In FIGS. 4A-6B, distal is to the right and proximal is to the left.

Any other feature from FIGS. 1A-1C may be included in FIGS. 2A-4B, and may in fact be expressly included and shown in FIGS. 2A-4B even if not expressly labeled. For example, the pump portions in FIGS. 2A-4B as shown include proximal and distal struts.

While FIGS. 2A-4B illustrates pump portions that include two impellers, each of which includes a tapering impeller blade region, any of these examples may be modified such that one of the impellers excludes a central tapering region, and the other has a constant or substantially constant diameter central region (such as the impellers in FIGS. 1A-1C). For example only, a distal impeller may have a constant diameter central region (optionally with tapering end region), while a proximal impeller may have a tapering central region (optionally with tapering end region), or vice versa.

When “central” is used in this context, it does not impart a requirement that the impeller have end regions that taper downward towards an axis; the impellers herein may have vertical proximal and distal ends, and can still be considered to have a tapering central region (e.g., FIGS. 2A-4B).

FIGS. 2A and 2B illustrate an exemplary pump portion 202 of catheter blood pump that includes an expandable blood conduit 212 and proximal and distal impellers 204 and 206. In this example the proximal impeller has a larger average diameter than the distal impeller, and has a greatest diameter that is greater than a greatest diameter of the distal impeller. In this example, the scaffold 210 tapers from a smaller diameter at the distal impeller to a larger diameter at the proximal impeller. As previously described, the scaffold can be attached or connected to a membrane or cover to form the blood conduit, which has a configuration with a distal to proximal taper that increases in diameter. This is an example of a configuration that is adapted to function as a diffuser between impellers to recover static pressure and increase efficiency. FIG. 2B illustrates an exemplary placement of the pump portion of the catheter blood pump of FIG. 2A across an aortic valve. The tapered configuration of the blood conduit is also adapted to create resistance across the aortic valve to resist distal movement or prevent the pump from moving distally further due to pump forces or accidental catheter manipulation. FIGS. 2A and 2B provide an example of a blood conduit with an outer profile configured to resist distal movement of the conduit when the pump is operational. The general outer profile of the blood conduit in FIGS. 2A and SB (as well as other blood conduits herein) increases in diameter from distal to proximal, which can decrease axial velocity and increase the static pressure.

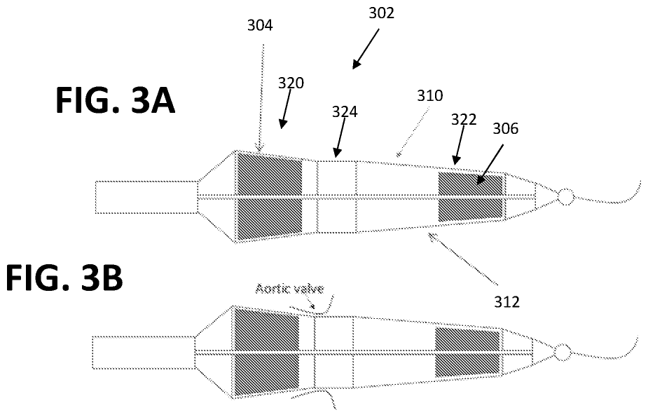

FIGS. 3A and 3B illustrate an exemplary pump portion 302 of a catheter blood pump including an expandable blood conduit 312 formed at least in part by a scaffold 310. The blood conduit includes a proximal tapered region 320 around a proximal impeller 304, and a distal tapered region 322, a portion of which surrounds a distal impeller 306 as shown. The blood conduit and/or scaffold includes a constant diameter transition section 324 (“straight) in between the tapered blood conduit sections in this example, as shown. In alternative examples, the constant diameter section may be replaced with a tapered section.

Unlike the embodiment of FIGS. 2A and 2B, the embodiment of FIGS. 3A and 3B illustrates multiple blood conduit tapered sections that are not part of a continuous tapering profile. The embodiment of FIGS. 3A and 3B is an example of a blood conduit that may be described as having a non-constant diameter and having an outer profile that is not any part of a cone shape with a circular base. In alternatives to FIGS. 3A and 3B, the blood conduit may include tapered sections along its length, but the entirety of the blood conduit profile may not be continuously tapered, such as if the different tapered regions have different characteristics, such as degree of the taper (e.g., gradual versus steeper). Having different blood conduit sections with varying outer profiles, such as is shown in FIGS. 3A and 3B, may provide more design options for optimizing pump performance. For example, the degree of a taper may be varied (e.g., for different impeller regions), a length of a straight section may be varied, etc. The outer profile of the blood conduit in FIGS. 3A and 3B is also adapted to create resistance across the aortic valve to resist distal movement or prevent the pump from moving distally further due to pump forces or accidental catheter manipulation. The embodiment of FIGS. 3A and 3B is an example of a blood conduit with an outer profile configured to resist distal movement of the conduit when the pump is operational, and may be thought of as having a plug configuration.

FIGS. 4A and 4B illustrate an exemplary pump portion 402 of a catheter blood pump that is similar to the pump portion in FIGS. 3A and 3B, and includes a blood conduit with a bend 426 preformed therein, exemplary features of which may be found in International Patent Application No. PCT/US2021/054238, filed on Oct. 8, 2021, which is incorporated by reference herein for all purposes. The bend 426 is in a central region of the blood conduit 412 between a distal impeller 406 and a proximal impeller 404, as shown. The exemplary bend as shown can be configured to increase anatomic geometric compatibility. For example, the shape of the exemplary pump portion in FIGS. 4A and 4B can help optimize pump geometry to accommodate the difference in aortic valve axis and a long axis of a left ventricle. For example, in methods of placement, the pump is positioned such that the proximal impeller (a rotation axis thereof) is substantially aligned with the aortic valve axis as shown, and the distal impeller (a rotational axis thereof) is substantially aligned with the long axis of ventricle. The bend can be configured to provide the respective axial alignments when the pump is positioned across an aortic valve. The configuration of the bend can also help prevent or minimize interaction between the pump portion distal end and the mitral valve apparatus, including chords thereof. The bend may also be adapted to avoid applying excessive pressure to regions of the ventricle, which could causing pacing issues. The outer profile of the blood conduit in FIGS. 4A and 4B is also adapted to create resistance across the aortic valve to resist distal movement or prevent the pump from moving distally further due to pump forces or accidental catheter manipulation. FIGS. 4A and 4B are an example of a blood conduit with an outer profile configured to resist distal movement of the conduit when the pump is operational.

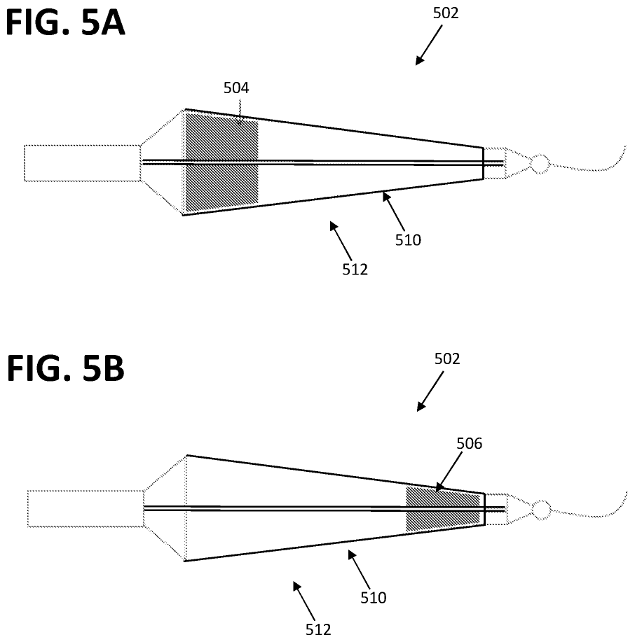

FIG. 5A illustrates one embodiment of a pump portion 502 that includes only a tapered proximal impeller 504 and a tapered expandable blood conduit 512 formed at least in part by a scaffold 510. As shown in this embodiment, the scaffold/blood conduit tapers from a larger diameter on the proximal end of the pump portion to a narrower or smaller diameter on the distal end of the pump portion. In this example, the taper of the blood conduit/scaffold matches or aligns with the taper of the proximal impeller 504.

In contrast, the embodiment of FIG. 5B illustrates one embodiment of a pump portion 502 that includes only a tapered distal impeller 506 and a tapered expandable blood conduit 512 formed at least in part by a scaffold 510. As shown in this embodiment, the scaffold/blood conduit tapers from a larger diameter on the proximal end of the pump portion to a narrower or smaller diameter on the distal end of the pump portion. In this example, the taper of the blood conduit/scaffold matches or aligns with the taper of the proximal impeller 504.

It should be understood that in some embodiments of the blood pump of FIGS. 5A-5B, the blood conduit/taper can taper from a larger diameter at the distal end to a smaller diameter at the proximal end. Additionally, as described and illustrated above and particularly in the embodiments of FIGS. 3A-3B, the blood conduit/scaffold can include sections that have a constant diameter (e.g., do not taper), particularly in the middle portion of the blood pump. In some embodiments, the blood pump can include a tapered proximal impeller with a matching taper in the blood conduit/scaffold, which extends into a constant diameter section of the blood conduit/scaffold until it reaches the distal end of the device. In other embodiments, the tapered section can include the distal portion that houses a tapered distal impeller, and the constant diameter section can extend proximally along the pump portion until the catheter. Any of the embodiments or features described herein, such as tapered sections, tapered impellers, constant diameter sections, etc., can be mixed and matched with other embodiments described herein.

In any of the embodiments herein, tip gaps may be the same for the first and second impellers. In any alternative, however, the tips gaps may be different. For example, a tip gap for the larger diameter impeller maybe larger than a tip gap for the smaller diameter impeller.

Additionally, in any of the embodiments herein, the proximal impeller may have a larger average diameter or greatest diameter larger than the distal impeller, and may generate more pressure (analogous to work) than a smaller distal impeller.

1. A catheter blood pump, comprising:
a pump portion that includes:
an impermeable blood conduit and first and second impellers at least partially disposed in the blood conduit,
wherein the first and second impellers have first and second average diameters, respectively, that are different.

2. The blood pump of claim 1, wherein the first impeller is a proximal impeller and has an average diameter that is larger than an average diameter of the distal impeller.

3. The blood pump of claim 1, wherein the first impeller is a distal impeller and the second impeller is a proximal impeller, and wherein the distal impeller has a diameter larger than a diameter of the proximal impeller.

4. The blood pump of claim 1, wherein the blood conduit includes a first section and a second section, the first section having a greater average diameter than an average diameter of the second section, wherein the first section at least partially surrounds the one of the first and second impellers with the greater average diameter.

5. The blood pump of claim 4, wherein the blood conduit includes a transition section between the first and second sections, the transition section having a varying diameter between the first and second section average diameters.

6. The blood pumps of claim 1, further comprising a plurality of expandable proximal struts extending proximally from the blood conduit.

7. The blood pumps of claim 1, further comprising a plurality of expandable distal struts extending distally from the blood conduit.

8. The blood pump of claim 1, wherein the first impeller has an average diameter that is between 100% and 500% of an average diameter of the second impeller.

9. The blood pump of claim 8, wherein the first impeller has an average diameter that is from 125% to 400% of an average diameter of the second impeller.

10. The blood pump of claim 1, wherein the blood conduit, first impeller, and second impeller are all configured to be expandable and collapsible.

11. The blood pump of claim 1, wherein the blood conduit, first impeller, and second impeller are not configured to be expandable and collapsible.

12. The blood pump of claim 1, wherein the first impeller is adapted and configured to be expanded and collapsed, and wherein the second impeller is adapted and configured such that is does not expand and collapse.

13. The blood pump of claim 12, further comprising a delivery sheath, the delivery sheath and the second impeller sized so that the delivery sheath is configured to cause the collapse of the second impeller when the delivery sheath is moved distally relative to the second impeller.

14. The blood pump of claim 1, wherein at least one of the first and second impellers, optionally both, includes at least one blade with a diameter that tapers in a tapering region, the tapering region including a location of an impeller greatest diameter, optionally wherein the greatest diameter is at an end of the tapering region.

15. The blood pump of claim 14, wherein a tip gap between an outer edge of the at least one blade and an inner wall of the blood conduit is constant in the tapering region.

16. The blood pump of claim 14, where the first impeller has its largest diameter in an impeller constant diameter region of the impeller, optionally wherein the first impeller is a proximal impeller, and optionally wherein the first impeller is distal impeller.

17. The blood pump of claim 1, wherein at least one of the first and second impellers, optionally both, includes at least one blade with a diameter that tapers in a tapering region, the tapering region including a location or region where an impeller blade is closest to a blood conduit inner wall.

18. The blood pump of claim 1, wherein at least one of the first and second impellers, and optionally both, have a greatest diameter in an impeller constant diameter region.

19. The blood pump of claim 5, wherein the transition section has a continuously tapering configuration.

20. The blood pump of claim 5, wherein the transition section comprises an outer profile with a configuration adapted to contact tissue and resist distal migration of the pump portion towards a left ventricle.

21-37. (canceled)
